(19) United States
(12) Patent Application Publication
(10) Pub. No.: US 2008/0044929 A1
(43) Pub. Date: Feb. 21, 2008

(54) Diluent, methods of manufacturing and use

(75) Inventors: John Lynch, Billerica, MA (US); Alan Weiss, Acton, MA (US)

Correspondence Address: MILLIPORE CORPORATION, 290 CONCORD ROAD, BILLERICA, MA, 01821, US

(73) Assignee: Millipore Corporation, Billerica, MA (US)

(21) Appl. No.: 11/975,768

(22) Filed: Oct. 22, 2007

Related U.S. Application Data
(62) Division of application No. 10/365,208, filed on Feb. 12, 2003.
(60) Provisional application No. 60/356,789, filed on Feb. 14, 2002.

(51) Int. Cl. G01N 33/536

(52) U.S. Cl. 436/536

(57) ABSTRACT

The use of diluent to reduce non-specific,drug binding (NSB) provides a simple, flexible and biocompatible way to reduce chemical entity (such as drugs, drug candidates and other small molecules) NSB so that bioassay results may more closely predict the behavior of these compounds in vitro. Additionally, the use of diluent as the chemical entity diluent enhances the predictive nature of data emanating from high throughput drug assays such as Caco-2 drug transport assays, plasma protein drug binding assays, PAMPA assays, permeability assays, and drug solubility assays. The diluent is made by either filtering a selected plasma through an ultrafiltration membrane having nominal molecular weight cutoff of about 30 kD, preferably about 10 kD or below or by selectively adding individual components of a plasma or serum that do not contribute to non-specific binding.

### CROSS-REFERENCED TO RELATED APPLICATIONS

This application is a divisional application of co-pending application Ser. No.: 10/365,208, filed on Feb. 12, 2003 which claims the benefit of Provisional Application No. 60/356,789, filed on Feb. 14, 2002.

The present invention relates to biocompatible diluent and a method for its manufacture and use. More particularly, it relates to a diluent to prevent non-specific binding of small molecules in in vitro chemical entity testing.

### BACKGROUND OF THE INVENTION

High throughput screening assays (e.g., Caco-2 drug transport, Parallel Artificial Membrane Permeability Assay [PAMPA], plasma protein drug binding, solubility testing, etc.) are done in multiple well devices, that are comprised of anywhere from 12 to 1536 distinct wells. These are used to identify and characterize various low molecular weight organic molecules (Chemical entities or “CEs”) that have or are believed to have some pharmaceutical use. Typically, CEs are investigated in drug discovery procedures to determine whether they are potentially viable drug candidates.

Frequently, the bottom of the wells of these plates, for example, the Caco-2 Multiscreen® plate, contain a membrane or filter, to facilitate or enable some aspect of the screening assay. In instances in which a filter or membrane-bottomed plate is used, there may also be a receiver tray that fits under the multi-well plate into which sample is filtered or into which the contents of the filter plate may diffuse. CEs may be introduced into these plates or may end up in the receiver plate volume at very low concentrations (<<1 micromolar). Being able to determine the absolute concentration of CEs in these dilute solutions is critical and non-specific binding losses of CEs to plastic surfaces, the membrane and other constituents of the testing fluid such as proteins could potentially limit the usefulness of these devices.

In addition to being present in solutions at low concentrations (<<1μM), many CEs are lipophilic and therefore have a tendency to be non-specifically adsorbed or bound (more commonly known as non-specific drug binding, hereafter “NSB”) onto the plastic surfaces such as the multi-well plate, membrane, receiver plates or other constituents of the testing solution such as proteins and seemingly disappear from the testing solutions. This loss of the CE can significantly affect the outcome or interpretation of the assay and lead to inaccurate and misleading results. As the surface area-to-volume ratio increases and the concentrations decrease, NSB issues become more likely.

Several attempts have been made to reduce NSB.

It is commonly believed that the choice of the plastic used in the receiver plate is important in terms of controlling NSB. Different types of mechanisms to mask the plastic surface have been tried as means to eliminate NSB.

The addition of relatively small amounts of organic solvent (e.g., DMSO, methanol, DMF, THF, etc.) has in some instances been found to significantly reduce levels of CE NSB, but these solvents also have the potential to alter the behavior of these CEs relative to plasma protein binding and their apparent permeability and therefore are generally unacceptable.

Another way to reduce NSB, especially to receiver plates, has been to precoat the plastic with a blocking agent. Proteins, such as BSA, have been found to be effective for some CEs, but there is a risk that they may bind the CEs and remove them from the assay leading to false results.

Another solution to minimize NSB has been to use a polyolefin blended with a small amount of PTFE or other low NSB polymer. One such receiver plate is a PTFE/polypropylene receiver plate used in connection with Microcon®) filter devices, both available from Millipore Corporation of Billerica, Mass. See U.S. Pat. No. 6,635,430 and U.S. Pat. No. 6,544,417.

The receiver plate works well for most CEs and drugs and represents a vast improvement over the receiver plates of the prior art. However, with low solubility or lipophilic CEs, even this receiver plate has been shown to have significant levels of NSB for a number of low solubility and/or lipophilic CEs.

Alternatively, one can use a plastic receiver plate that has been precoated with a hydrophilic polymer to reduce NSB. This solution appears to be fundamentally sound, but it has at least two serious limitations. The first is that these surface treated receiver plates—at least as they are currently provided—have severe dimensional constraints. Receiver plates vary in size and format depending upon a number of variables such as the design and format of the top plate, the test being conducted, the individual manufacturer's designs and preferences and the like. As such, they cannot be handled by robotic laboratory equipment and are not compatible with automated high throughput screening techniques. Secondly, these coated receiver plates provide no protection against NSB on the other surfaces in the device (e.g., the top plate, the membrane, the flow director, etc.) or the testing solution itself.

In order to make these types of assays more predictive of in vitro behavior, some more universal means to prevent or reduce NSB is needed.

### SUMMARY OF THE INVENTION

The use of a diluent to reduce non-specific drug binding (NSB) provides a simple, flexible and biocompatible way to reduce CE non specific binding (NSB) such as in drug and drug candidate (and other small molecule) testing so that bioassay results may more closely predict the behavior of these compounds in vitro. Moreover, it provides the benefit against NSB throughout the test apparatus, not just in one of the components. This provides a universal solution to the issue of NSB and allows one to use any plate design with any test as one may desire. The use of plasma or serum diluent or a synthetic diluent as taught by the present invention as the CE diluent enhances the predictive nature of data emanating from high throughput CE assays such as Caco-2 drug transport assays, plasma protein drug binding assays, PAMPA assays, permeability assays, and drug solubility assays.

It is an object of the present invention to provide a biocompatible diluent for performing a range of chemical entity (CE) assays including cell based and non-cell based assays that utilities a diluent, wherein the diluent has been produced by filtering serum or plasma through a membrane with a nominal molecular weight cut-off of 50 kD, preferably 30 kD or below.

It is an additional object of the present invention to provide a method of forming a diluent comprising the steps of selecting a source material selected from the group consisting of individual components of plasma, serum and blends thereof, blending the individual components in a buffered physiological saline solution to form the diluent.

It is another object of the present invention to provide a diluent for reducing non-specific binding of chemical entities in a biological test system comprising an diluent, wherein the diluent has a nominal molecular weight of below 50 kD, is formed from a source material selected from the group consisting of plasma, serum and blends thereof and is formed by a process elected from the group consisting of filtering the source material through a filter having a nominal molecular weight cutoff of less than about 50 kD or by blending individual components of the source material having a nominal molecular weight of less than about 50 kD in a buffered physiological saline solution and wherein the diluent is biocompatible and maintains the drug or small molecule's solubility and bioavailability properties.

It is a further object of the present invention to provide a method of forming a diluent comprising the steps of selecting a source material formed of one or more plasma or sera or blends thereof, filtering the source material through an ultrafiltration membrane having a nominal molecular weight cutoff of about 30 kD, preferably about 10 kD and recovering the filtrate from the filtration step.

It is another object of the present invention to provide a process for reducing the non-specific binding of chemical entities (CEs) comprising selecting a CE to be tested and a method by which the CE will be tested, selecting a multi-well plate having one or more wells, performing the assay in the one or more wells of the multi-well plate, diluting the CE with the diluent that has low non specific binding for the CE to a desired concentration, applying the diluted CE to the plate in the one or more wells of the multi-well plate and collecting and analyzing the CE.

It is a further purpose of the present invention to provide a process for the testing of CEs such as drug candidates comprising selecting a CE to be tested, selecting a testing device having one or more wells, each well having a bottom closed by a porous structure, positioning the device over a collection device comprised of one or more wells, each well having an open top and a closed bottom and being in register with a well of the testing device so as to receive filtrate from the one or more wells of the testing device, diluting the CE with diluent to a desired concentration and applying the diluted CE to the one or more wells of the testing device, capturing the filtrate of the testing device in one or more wells of the receiving device and collecting and analyzing the filtrate for drug activity.

It is an additional object of the present invention to provide a diluent for reducing non-specific binding of CEs such as drugs and other small molecules in a biological test system comprising an diluent, wherein the diluent has a nominal molecular weight of below 30 kD, preferably below 10 kD and is formed from a source material selected from the group consisting of plasma, serum and blends thereof and wherein the diluent is biocompatible and maintains the drug or small molecule's solubility and bioavailability properties.

It is another object of the present invention to provide a diluent for reducing non-specific binding of CEs such as drugs and other small molecules in a biological test system comprising a diluent, wherein the diluent is formed of individual sterile components of plasma, serum and blends thereof and wherein the diluent is biocompatible and maintains the CE's solubility and bioavailability properties.

### IN THE DRAWINGS

FIG. 1 shows a device useful in one embodiment of the present invention in cross section.

FIG. 2 shows data from the Example in graphical form.

FIG. 3 shows data from the Example in graphical form.

### DETAILED DESCRIPTION OF THE INVENTION

The present invention relates to a diluent for chemical entities (CEs). More particularly, it relates to the diluent and its use as the preferred diluent for reducing or eliminating non-specific binding (NSB) to test devices surfaces and fluids used to measure compound transport, solubility, adsorption, distribution, binding and other compound properties of chemical entities in an assay predictive of in vitro compound behavior.

The diluent is an aqueous solution comprised of low molecular weight constituents that either do not contribute to NSB or which eliminate NSB of chemical entities and which have little or no adverse impact on the analysis of the CEs and which do not interfere with the methods and assays for the determination and quantification of such CEs.

The diluent may either be formed from a native source such as plasma, serum and the like through selective filtration to remove components that add to NSB or by mixing individual components of the native sources to create such a diluent.

The diluent is typically formed from plasma or serum that has been subjected to one or more filtration steps, mainly ultrafiltration steps, such that the plasma or serum essentially contains little if no proteins and other components that contribute to non specific binding of chemical entities (CEs). Preferably, it is a fluid that has essentially all constituents of a nominal molecular weight of about 50 kiloDaltons (kD) or below, more preferably it contains constituents that have a nominal molecular weight of less than 30 kiloDaltons (kD), even more preferably it contains constituents that have a nominal molecular weight of less than 10 kiloDaltons (kD) and which is essentially protein-free. If desired, even finer cuts of diluent may be used such as a nominal molecular weight of 5 kD or less. However, for most applications, diluent having a nominal molecular weight of less than 30 kD is acceptable and provides all of the expected benefits.

Alternatively, a solution may be made by mixing various low molecular weight constituents of serum or plasma such as salts, triglycerides, cholesterols, sugars and the like (kD below 50, preferably below 30 kD, more preferably below 10) to buffered physiological saline solution to form the diluent. The components are well known in the art and they are available from various commercial sources and can also be easily separated by one of ordinary skill in the art in a laboratory using common techniques and equipment. (See for example, Table 1).

TABLE 1

| | | CONVENTIONAL | | RECOMMENDED |
|---|---|---|---|---|
| COMPONENT | SYSTEM | UNITS | FACTOR | SI UNITS |
| Acetoacetic Acid | | | | |
| Qualitative | Serum | Negative | — | Negative |
| Quantitative | Serum | 0.2-1.0 mg/dL | 97.95 | 20-100 μmol/L |
| Acetone | | | | |
| Qualitative | Serum | Negative | — | Negative |
| Quantitative | Serum | 0.3-2.0 mg/dL | 172.95 | 20-340 μmol/L |
| Alcohol, ethyl | Serum or Whole Blood | Negative - but presented | 0.2171 | Negative - but |
| | | as mg/dL | | present as mmol/L |
| δ-Aminolevulinic acid | Serum | 0.01-0.03 mg/dL | 76.26 | 0.8-2.3 μmol/L |
| Ammonia | Plasma | 20-120 μg/dL(diffusion) | 0.5872 | 12-70 μmol/L |
| | | 40-80 μg/dL (enzymatic | | 23-47 μmol/L |
| | | method) | | |
| | | 12-48 μg/dL (resin | | 7-28 μmol/L |
| | | method) | | |
| Arsenic++ | Whole blood | <7 μg/dL | 0.05055 | <0.4 μmol/L |
| Ascorbic Acid (vitamin C) | Plasma | 0.6-1.6 mg/dL | 56.78 | 34-91 μmol/L |
| | Whole blood | 0.7-2.0 mg/dL | | 40-114 μmol/L |
| Bicarbonate | Plasma | 21-28 mmol/L | 1 | 21-28 mmol/L |
| Bilirubin | | | | |
| Direct (conjugated) | Serum | <0.3 mg/dL | 17.10 | <5 μmol/L |
| Indirect (unconjugated) | | 0.1-1.0 mg/dL | | 2-17 μmol/L |
| Total | | 0.1-1.2 mg/dL | | 2-21 μmol/L |
| Newborns Total | | 1.0-12.0 mg/dL | | 17-205 μmol/L |
| Blood Gases | Whole Blood | 7.38-7.44 (arterial) | 1 | 7.38-7.44 |
| PH | Whole Blood | 7.36-7.41 (venous) | 0.1333 | 7.36-7.41 |
| Pco2 | Whole Blood | 35-40 mm Hg (arterial) | 0.1333 | 4.7-5.3 kPa |
| Po2 | | 40-45 mm Hg (venous) | | 5.3-6.0 kPa |
| | | 95-100 mm Hg (arterial) | | 12.7-13.3 kPa |
| Bromide | Serum | <5 mg/dL | 0.125 | <0.63 mmol/L |
| Calcium | Serum | 4.0-4.8 mg/dL | 0.2500 | 1.00-1.20 mmol/L |
| Ionized | Serum | 2.0-2.4 mEq/L | 0.5000 | 0.30-1.58 of total |
| Total | | 30-58% of total | 0.01 | 2.30-2.74 mmol/L |
| | | 9.2-11.0 mg/dL | 0.2500 | |
| | | 4.6-5.5 mEq/L | 0.5000 | |
| Carbon Dioxide (CO2 | Whole Blood (arterial) | 19-24 mmol/L | 1 | 19-24 mmol/L |
| content) | Plasma or Serum | 21-28 mmol/L | | 21-28 mmol/L |
| | (arterial) | | | |
| Carbon Dioxide | Whole blood (venous) | 22-26 mmol/L | 1 | 22-26 mmol/L |
| | Plasma or | 24-30 mmol/L | | 24-30 mmol/L |
| | Serum (venous) | | | |
| CO2 combining power | Plasma or | 24-30 mmol/L | 1 | 24-30 mmol/L |
| | Serum (venous) | | | |
| CO2 partial pressure (Pco2) | Whole Blood (arterial) | 35-40 mm Hg | 0.1333 | 4.7-5.3 kPa |
| | Whole Blood (venous) | 40-45 mm Hg | | 5.3-6.0 kPa |
| Carbonic acid (H2CO3) | Whole Blood (arterial) | 1.05-1.45 mmol/L | 1 | 1.05-1.45 mmol/L |
| | Whole Blood (venous) | 1.15-1.50 mmol/L | | 1.15-1.50 mmol/L |
| | Plasma (venous) | 1.02-1.38 mmol/L | | 1.02-1.38 bmmol/L |
| Carotene beta | Serum | 40-200 Fg/dL | 0.01863 | 0.73.7 μmol/L |
| Chloride | Serum | 95-103 mEq/L | 1 | 95-103 mmol/L |
| Cholesteral | Serum | 150-250 mg/dL (varies | 0.02586 | 3.88-6.47 mmol/L |
| Total | Serum | with diet, sex, and age) | 0.01 | Fraction of total |
| Esters | | 65-75% of total | | cholesterol: 0.65-0.75 |
| | | cholesterol | | |
| Citrate | Serum or Plasma | 1.7-3.0 mg/dL | 52.05 | 88-156 μmol/L |
| Copper | Serum or Plasma | 70-140 Fg/dL | 0.1574 | 11-22 μmol/L |
| | Male | 80-155 Fg/dL | | 13-24 μmol/L |
| | Female | | | |
| Cortisol | Plasma | 5-23 Fg/dL | 27.59 | 138-635 nmol/L |
| | 8am-10am | 3-13 Fg/dL | | 83-359 nmol/L |
| | 4pm-6pm | | | |
| Creatine | Serum or Plasma | 0.1-0.4 mg/dL | 76.25 | 8-31 μmol/L |
| | Male | 0.2-0.7 mg/dL | | 15-53 μmol/L |
| | Female | | | |
| Creatine kinase (CK) | Serum | 55-170 U/L at 37EC | 1 | 55-170 U/L at 37EC |
| | Male | 30-135 U/L at 37EC | 1 | 30-135 U/L at 37EC |
| | Female | | | |
| Creatinine | Serum or Plasma | 0.6-1.2 mg/dL (adult) | 88.40 | 53-106 μmol/L |
| | | 0.3-0.6 mg/dL (children | | 27-53 μmol/L |
| | | <2y) | | |
| Fats, nuetral (see | | | | |
| Triglycerides) | | | | |
| Fatty Acids | Serum | 9-15 mmol/L | 1 | 9-15 mmol/L |
| Total (free and esterified) | Plasma | 300-480 FEq/L | 1 | 300-480 μmol/L |
| Free (nonesterified) | | | | |
| Fluoride | Whole Blood | <0.05 mg/dL | 0.5263 | <0.027 mmol/L |
| Folate | Serum | 5-25 ng/mL (bioassay) | 2.266 | 11-57 nmol/L |
| | Erythrocytes | >2.3 ng/mL (radioassay) | | >5 nmol/L |
| | | 166-640 ng/mL | | 376-1450 nmol/L |
| | | (bioassay) | | >317 nmol/L |
| | | >140 ng/mL (radioassay) | | |
| Galactose | Whole Blood | None | 0.05551 | None |
| | Adults | <20 mg/dL | | <1.11 mmol/L |
| | Children | | | |
| Glucose, fasting | Serum or Plasma | 70-110 mg/dL | 0.05551 | 3.9-6.1 mmol/L |
| | Whole Blood | 60-100 mg/dL | | 3.3-5.6 mmol/L |
| Glutathione | Whole Blood | 24-37 mg/dL | 0.03254 | 0.78-1.20 mmol/L |
| Growth hormone | Serum | <10 ng/mL | 1 | <10 μg/L |
| 17-Hydroxycorticosteroids | Plasma | | 25.591 | |
| | Male | 7-19 Fg/dL | | 193-524 nmol/L |
| | Female | 9-21 Fg/dL | | 248-579 nmol/L |
| | After 24 USP | 35-55 Fg/dL | | 966-1517 nmol/L |
| | Units of ACTH | | | |
| | I.M. | | | |
| Iron, total | Serum | 60-150 Fg/dL | 0.1719 | 10.7-26.9 μmol/L |
| 17-Ketosteroids | Plasma | 25-125 Fg/dL | 34.67W | 866-4334 nmol/L |
| Lactic acid (as lactate) | Whole Blood | | 0.1110 | |
| | Venous | 5-20 mg/dL | | 0.6-2.2 mmo/L |
| | Arterial | 3-7 mg/dL | | 0.3-0.8 mmol/L |
| Lead | Whole blood | <50 Fg/dL | 0.04826 | <2.41 μmol/L |
| Lipids, total | Serum | 400-800 mg/dL | 0.01 | 4.00-8.00 g/L |
| Cholesterol | | 150-250 mg/dL | 0.02586 | 3.88-6.47 mmol/L |
| Triglycerides | | 10-90 mg/dL | 0.01129** | 0.11-2.15 mmol/L |
| Phospholipids | | 150-380 mg/dL | 0.01 | 1.50-3.80 g/L |
| Fatty acids (free) | | 9.0-15.0 mmol/L | 1 | 9.0-15.0 mmol/L |
| Phospholipid phosphorus | | 8.0-11.0 mg/dL | 0.3229 | 2.58-3.55 mmol/L |
| Oxygen | Whole Blood (arterial) | 95-100 mm Hg | 0.1333 | 12.7-13.3 kPa |
| Pressure (Po2) | Whole Blood (arterial) | 15-23 volume % | 0.01 | Volume |
| Content | Whole Blood (arterial) | 94-100% | | fraction: 0.15-0.23 |
| Saturation | | | | Fraction |
| | | | | saturated: 0.94-1.00 |
| Phenylalanine | Serum | | 60.54 | |
| | Adults | <3.0 mg/dL | | <182 μmmol/L |
| | Newborns (term) | 1.2-3.5 mg/dL | | 73-212 μmmol/L |
| Phosphorus, inorganic | Serum | | 0.3229 | |
| | Adults | 2.3-4.7 mg/dL | | 0.74-1.52 mmol/L |
| | Children | 4.0-7.0 mg/dL | | 1.29-2.26 mmol/L |
| Potassium | Plasma | 3.8-5.0 mEq/L | 1 | 3.8-5.0 mmol/L |
| Protoporphyrin | Erythrocytes | 15-50 mg/dL | 0.01777 | 0.27-0.89 μmol/L |
| Pyruvate | Whole Blood | 0.3-0.9 mg/dL | 113.6 | 34-102 μmol/L |
| Salicylates | Serum | Negative | — | negative |
| Therapeutic interval | | 15-30 mg/dL | 0.07240 | 1.08-2.17 mmol/L |
| Sodium | Plasma | 136-142 mEq/L | 1 | 136-142 mmol/L |
| Sulfate, inorganic | Serum | 0.2-1.3 mEq/L | 0.5 | 0.10-0.65 mmol/L |
| | | 0.9-6.0 mg/dL as SO4 | 0.1042 | 0.09-0.63 mmol/L |
| | | | | as SO4 |
| Testosterone | Serum or Plasma | | | |
| | Male | 300-1200 ng/dL | 0.03467 | 10.4-41.6 nmol/L |
| | Female | 30-95 ng/dL | | 1.0-3.3 nmol/L |
| Triglycerides | Serum | 10-190 mg/dL | 0.01129** | 0.11-2.15 mmol/L |
| Urea nitrogen | Serum | 8-23 mg/dL | 0.357 | 2.9-8.2 mmol/L |
| Uric acid | Serum | 4.0-8.5 mg/dL | 0.05948 | 0.24-0.51 mmol/L |
| | Male | 2.7-7.3 mg/dL | | 0.16-0.43 mmol/L |
| | Female | | | |
| Vitamin A | Serum | 15-60 Fg/dL | 0.03491 | 0.52-2.09 μmol/L |
| Vitamin B12 | Serum | 160-950 pg/ml | 0.7378 | 118-701 pmol/L |
| Vitamin C | Plasma | 0.6-1.6 mg/dL | 56.78 | 34-91 μmol/L |
| Zinc | Serum | 50-150 Fg/dL | 0.1530 | 7.7-23.0 μmol/L |

The plasma or serum or the individual components selected can be from a variety of sources, including but not limited to bovines such as cattle and fetal calf serum, sheep, goat, human plasma and serum, protein-free serum products and protein-free, animal-free serum products and the like. These are available from a variety of vendors such as Sigma Aldrich, Hyclone Inc. and Gibco/Invitrogen.

The native sourced diluent is made by selecting a desired source material, be it one or more plasma sources or serum sources or blend of one or more plasmas, sera or both and subjecting the source material to one or more filtration steps with at least one step being the filtration of the source material through an ultrafiltration membrane having a nominal molecular weight cutoff of about 50 kD, or 30 kD or 10 kD. Coarser prefilters may be used before this ultrafiltration step particularly if the plasma or serum has a large amount of larger molecular weight constituents that would otherwise clog or foul the ultrafilter. Further, if desired, one can use additional ultrafiltration steps to create even finer products if desired or necessary for the particular application.

The ultrafiltration step may occur in a normal flow filter such as a Centricon® centrifugal filter device, or an ultrafiltration membrane such as a PLGC UF membrane (10 kD N.M.W.C.O.), a YMT10 membrane (N.M.W.C.O. of 10 kD) or a PLTK UF membrane (30 kD N.M.W.C.O.) contained in a stainless steel filter holder or in a SWINNEX® filter holder (all available from Millipore Corporation of Billerica, Mass.), a stirred cell, a tangential flow filter device such as a Pellicon® UF cassette containing a PLTK or PLGC UF membrane or through a hollow fiber device, such as is shown in U.S. Pat. No. 5,626,758. The system selected is not critical to the application and has more to do with the scale/volume of source material to be filtered as well as the existing equipment one has at hand.

A preferred method is to use Fetal Bovine Serum as the source material and clarify it in a Stericup® filter device available from Millipore Corporation of Billerica, Mass. The diluent was prepared by separation of the clarified serum using a Millipore stirred cell fitted with an Ultracel® PLGC or an YMT10 membrane (N.M.W.C.O. of 10 kD), both available from Millipore Corporation of Billerica, Mass.

There are a number of different assays used to investigate and/or develop CEs as drug candidates and the like. The use of test cells, such as Caco 2 cells and the like may be used to test the intestinal transport properties of the CE. Others such as plasma protein binding, solubility testing, PAMPA, and other ‘artificial’ membrane transport (or permeability) assays do not require the use of cells. It is meant by this invention to provide a diluent for use in either type of test.

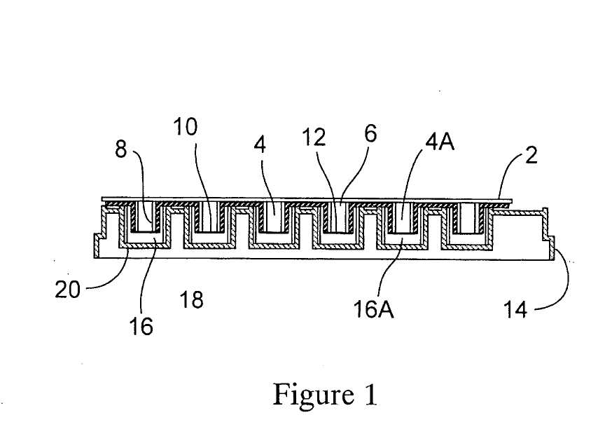

A typical assay comprises using a device similar to that shown in FIG. 1. This embodiment can be used with cell-based assays. Non-cell based assays might use a similar system with no cells contained within the system. The system comprises a top or cell plate 2 which has a series of wells, 4, typically 12, 24, 48 or 96 in number although lesser or greater numbers (such as 384 or 1536 wells) may be used.

The tops 6 of the wells are open and the bottoms 8 are closed by either a solid bottom or a porous structure 10, typically a microporous membrane or a glass filter. The porous structure 10 is sealed to the plate well bottoms such that cells and/or added constituents whose size exceeds the size of the membrane or filter's largest pore or which are retained by surface tension in the lack of a driving force for the filtration are retained within the wells and only liquid passes through the porous structure 10 by diffusion or under pressure. Cells 12 are grown on the upper surface of the porous structure 10 so that they form an integral layer I across the upper surface of the porous structure 10.

With the filter plate use, a receiver plate 14 is positioned below the cell plate 2. The receiver plate 14 has a series of wells 16 having an open top 18 and a closed bottom 20. The number of wells, their size and configuration are designed to register with those of the cell plate such that all liquid leaving a well 4A of the cell plate 2 flows into a respective well 16A of the receiver plate 14. In some non-cell assays, no receiver plate is necessary.

A chemical entity is diluted in the diluent of the present invention to a concentration believed appropriate for in vitro administration. Typically, the CE is diluted in the diluent to a level of from about 10 micromolar (μM) to about 0.1 nanomolar (nM) depending on the assay and CE being tested.

The CE in the diluent is then added to the open top of the wells 4 of the cell plate, preferably along the side of the wells 4 so as to not disturb the cells and allowed to interact with the cells. Preferably the wells 16 of the receiver plate 14 are filled with diluent (but containing no CE) as well.

After a time, typically an hour or so, the two plates 2,14 are separated and the liquid in the wells 16 of the receiver plate 14 are analyzed.

The diluent reduces the likelihood of any NSB of the CE to any of the test surfaces or fluids. Additionally, as it is a natural product and similar to the liquid that the cells are grown in, it has little if any adverse effect on the behavior of the cells or the CE, unlike other prior art methods such as the use of solvents. Moreover, as it is present through out the test system it reduces NSB not only in the receiver plate wells but also in the cell plate 2, the dilution vessel (not shown) the applicator such as a syringe or a pipette, the porous structure and the like. It has been found that the present invention works regardless of the materials used in the system, be they glass or plastic, blends of plastic or plastics coated with a hydrophilic coating and has been found to reduce NSB even in plates that were considered to be low NSB plates. Finally, the diluent will not bind any of the CE in solution meaning that there will be no negative impact on the CE's bioavailability.

The invention of the present invention has also been found to be the preferred diluent for materials used in a wide variety of assays, as it most closely resembles the in vitro environment. The invention may be used as a buffer, base media or diluent for different compounds used to assess compound behavior in biological and biopredictable assays.

### EXAMPLE

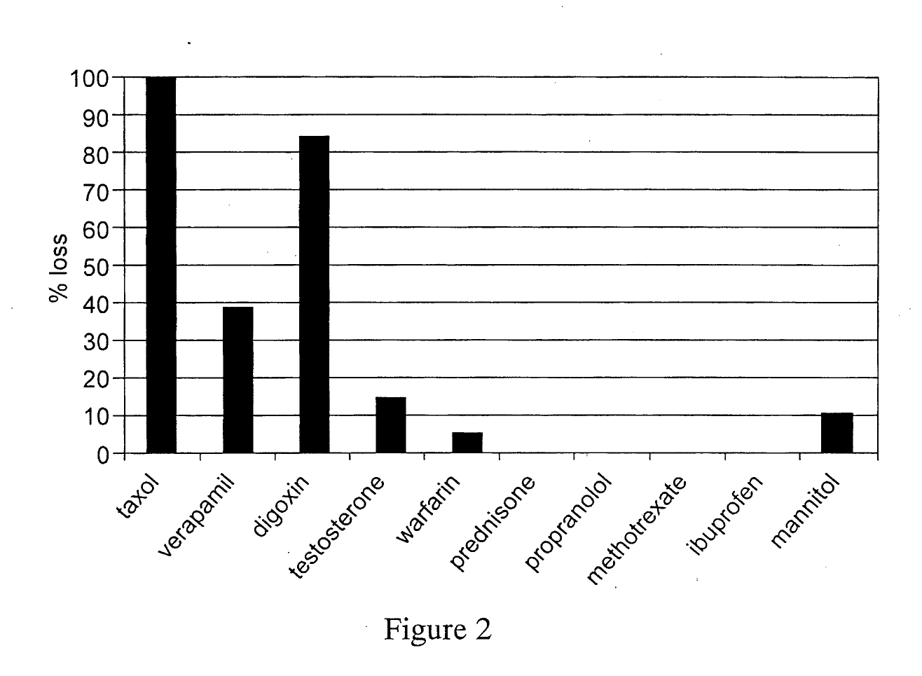

The NSB of various drugs (at 10 nM concentrations in phosphate buffered saline [PBS]) that had been radio labeled were added to a Microcon®96 receiver plate (formed of PTFE resin polypropylene blend) and left in the plate for 60 minutes. The amount of drug lost to NSB was measured and is summarized in FIG. 2.

The drug NSB (10 nM drug in phosphate buffer) to other 96-well plates made from different plastics was also tested. Binding was significant for all of these plates, including PTFE, with the extent of loss correlating with solubility of the drug (i.e., the more lipophilic the drug, the higher the loss due to NSB). In fact, drug NSB appears to be independent of plate material in a time course study for taxol and testosterone on PP and PTFE plates using LC-MS (data not shown).

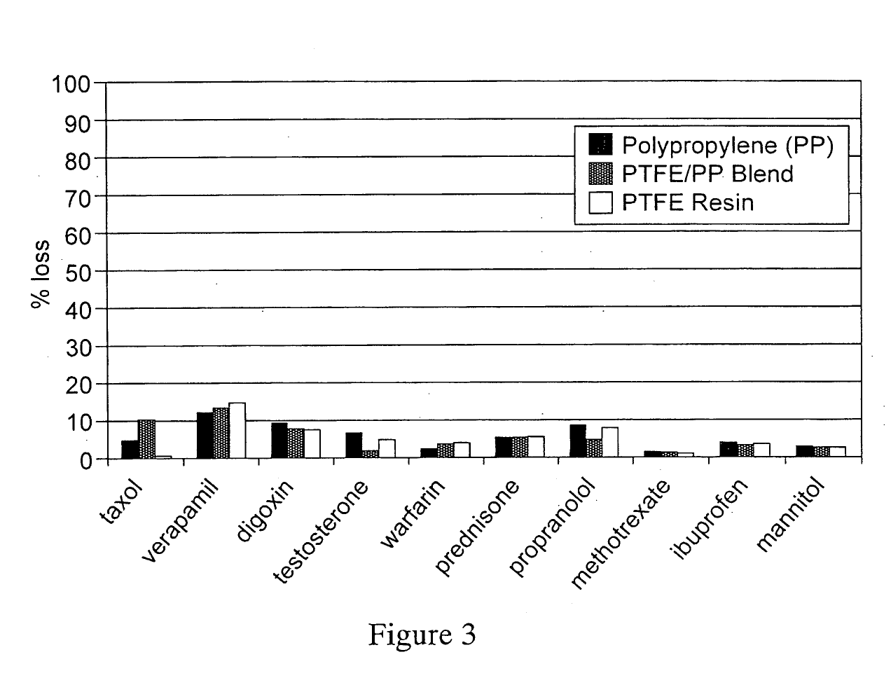

The dilution of drugs in diluent as claimed in the present invention was tested in the Microcon® 96 receiver plate (PTFE resin/polypropylene blend), a 96 well plate formed of polypropylene and a 96 well plate formed of PTFE resin with a variety of drugs and all showed significantly reduced NSB. The results are presented in FIG. 3.

In addition to the dramatic reduction observed in drug NSB as a consequence of making the dilutions in diluent, it appeared that diluent was also an ideal diluent for a wide range of assays since the diluent most closely resembles the in vivo ‘mobile phase’.

What is claimed:

1) A process for the testing of chemical entities with reduced non-specific binding of the chemical entities comprising selecting a chemical entities to be tested, selecting a testing device having one or more wells, diluting the chemical entity with a diluent to a desired concentration and applying the diluted chemical entity to the one or more wells of the testing device and collecting and analyzing the material wherein the diluent is derived from a source material selected from the group consisting of plasma, serum and blends thereof and is formed by a process elected from the group consisting of filtering the source material through a filter having a nominal molecular weight cutoff of less than about 50 kD or by blending individual components of the source material having a nominal molecular weight of less than about 50 kD in a buffered physiological saline solution.

2) The process for reducing the non-specific binding of chemical entities of claim 1 wherein each well has a bottom closed by a porous structure and further comprising a receiver plate positioned below the test device, the one or more wells of the receiver plate being in register with the one or more wells of the testing device, positioning the testing device over a receiver plate for receiving the filtrate from the one or more wells of the testing device into the one or more wells of the receiver plate, capturing the filtrate of the testing device in one or more wells of the receiver plate and collecting and analyzing the filtrate.

3) The process for reducing the non-specific binding of chemical entities of claim 1 wherein each well has a bottom closed by a porous structure and further comprising a cell line on which the chemical entity will be tested formed on the porous structure inside one or more of the wells of the testing device and a receiver plate positioned below the testing device, the one or more wells of the receiver plate being in register with the one or more wells of the testing device, positioning the testing device over a receiver plate for receiving the filtrate from the one or more wells of the testing device into the one or more wells of the receiver plate, capturing the filtrate of the testing device in one or more wells of the receiver plate and collecting and analyzing the filtrate.

4) The process of claim 1 wherein the test is selected from the group consisting of Caco-2 drug transport assays, plasma protein drug binding assays, PAMPA assays, permeability assays, and drug solubility assays.

5) A process for reducing the non-specific binding of a chemical entity to test equipment comprising the use of a diluent derived from a source material selected from the group consisting of plasma, serum and blends thereof and is formed by a process selected from the group consisting of filtering the source material through a filter having a nominal molecular weight cutoff of less than about 50 kD or by blending individual components of the source material having a nominal molecular weight of less than about 50 kD in a buffered physiological saline solution, adding the diluent to a chemical entity to achieve an in vivo dilution and applying the diluted chemical entity to a test assay.
